(19) United States
(12) Patent Application Publication
(10) Pub. No.: US 2026/0315579 A1
(43) Pub. Date: Oct. 15, 2026

(54) CARBONIC ANHYDRASE 4 AS A TARGET FOR THE TREATMENT OF LUNG FIBROSIS AND EMPHYSEMA

(71) Applicant: RUTGERS, THE STATE UNIVERSITY OF NEW JERSEY, New Brunswick, NJ (US)

(72) Inventor: Mark C. SIRACUSA, New Brunswick, NJ (US)

(73) Assignee: RUTGERS, THE STATE UNIVERSITY OF NEW JERSEY, New Brunswick, NJ (US)

(21) Appl. No.: 19/483,894

(22) PCT Filed: May 24, 2024
(86) PCT No.: PCT/US2024/031013

§ 371 Date: Nov. 12, 2025

Related U.S. Application Data
(60) Provisional application No. 63/468,725, filed on May 24, 2023.

(51) Int. Cl. A61K 38/51; A61K 9/00; A61K 31/198; A61K 31/4172; A61K 45/06; A61K 48/00; A61P 11/00; C12N 15/86

(52) U.S. Cl. CPC A61K 38/51; A61K 9/007; A61K 31/198; A61K 31/4172; A61K 45/06; A61K 48/005; A61P 11/00; C12N 15/86; C12Y 402/01001

(57) ABSTRACT

The invention provides methods that are useful for treating lung fibrosis and/or emphysema. In certain embodiments, the methods comprise administering to a mammal in need thereof (e.g., a human patient in need thereof) an effective amount of a Card enzyme. In certain embodiments, the methods comprise administering to a mammal in need thereof an effective amount of nucleic acid encoding a Card enzyme. Certain embodiments of the present invention also provide methods for treating lung fibrosis and/or emphysema comprising administering to a mammal in need thereof an effective amount of a Card enzyme activator, such as L-Histidine, D-Histidine, L-Phenylalanine, D-Phenylalanine, or a pharmaceutically acceptable salt thereof.

### CROSS-REFERENCE TO RELATED APPLICATION

This application claims priority to U.S. Provisional Application No. 63/468,725 that was filed on May 24, 2023. The entire content of the application referenced above is hereby incorporated by reference herein.

### BACKGROUND

Lung diseases including asthma, chronic obstructive pulmonary disease (COPD), idiopathic pulmonary fibrosis (IPF), emphysema and pneumonia represent the leading causes of death and disability in the world (Collaborators GBDCRD (2020) Lancet Respir Med. 8(6): 585-96; Societies FoIR (2017), The Global Impact of Respiratory Disease-Second Addition, Sheffield, European Respiratory Society). Unfortunately, the prevalence of pulmonary fibrosis, emphysema, and other lung-related pathologies are expected to see sharp increases as a result of the COVID-19 pandemic and the long-term consequences of SARS-COV-2 infection (George P M et al. (2020) Lancet Respir Med. 8(8): 807-15; Halpin D M G et al. (2021) Am J Respir Crit Care Med. 203 (1): 24-36). Despite these alarming facts, many lung diseases remain difficult to treat and have extremely poor outcomes. It is imperative that we develop a better understanding of the cellular and molecular mechanisms that regulate immunity and inflammation in the lung to inform therapeutic strategies to treat these devastating diseases. Alveolar macrophages (AMs) represent the most abundant immune cell in the parenchyma of the lung and, as such, they play diverse homeostatic and immunoregulatory roles (Kulikauskaite J et al (2020) Trends Immunol. 41(10): 864-77; Hussell T et al. (2014) Nat Rev Immunol. 14(2): 81-93; Hu G et al. (2019) Front Immunol. 10:2275). Despite their importance in the context of lung disease, the molecular mechanisms that regulate AM development and activation remain to be fully defined. Thus, a better understanding of these pathways is needed to aid in the development of novel therapeutic strategies and improved treatment options for diverse forms of lung inflammation.

### SUMMARY OF CERTAIN EMBODIMENTS OF THE INVENTION

Accordingly, certain embodiments of the invention provide a method for treating lung fibrosis and/or emphysema comprising administering to a mammal in need thereof an effective amount of at least one of a carbonic anhydrase 4 (Car4) enzyme, a nucleic acid encoding a Car4 enzyme, or a Car4 enzyme activator. In certain embodiments, a two- or three-way combination selected from a Car4 enzyme, a nucleic acid encoding a Car4 enzyme, and a Car4 activator, is administered to the mammal. In certain embodiments, the method is for treating lung fibrosis. In certain embodiments, the method is for treating emphysema.

Certain embodiments of the invention provide a kit comprising at least one of a Car4 enzyme, a nucleic acid encoding a Car4 enzyme, or a Car4 enzyme activator; packaging material; and instructions for administering the at least one of a Car4 enzyme, a nucleic acid encoding a Car4 enzyme, a Car4 enzyme activator to a mammal in need thereof to treat lung fibrosis and/or emphysema. In certain embodiments, the kit comprises two or more of a Car4 enzyme, a nucleic acid encoding a Car4 enzyme and a Car4 enzyme activator.

Certain embodiments of the invention provide a Car4 enzyme, a nucleic acid encoding a Car4 enzyme, a Car4 enzyme activator, or a combination thereof, for use in the treatment of lung fibrosis and/or emphysema. For example, certain embodiments of the invention provide 1) a Car4 enzyme and a nucleic acid encoding a Car4 enzyme; 2) a Car4 enzyme and Car4 enzyme activator; 3) a nucleic acid encoding a Car4 enzyme and Car4 enzyme activator; or 4) a Car4 enzyme, a nucleic acid encoding a Car4 enzyme, and a Car4 enzyme activator, for use in combination in medical therapy (e.g., for use in combination in treating lung fibrosis and/or emphysema).

Certain embodiments of the invention provide the use of a Car4 enzyme, a nucleic acid encoding a Car4 enzyme, a Car4 enzyme activator, or a combination thereof (e.g., a combination described herein), to prepare a medicament for the treatment of lung fibrosis and/or emphysema in a mammal in need thereof. For example, certain embodiments of the invention also provide the use of a Car4 enzyme to prepare a medicament useful for treating lung fibrosis and/or emphysema in combination with a nucleic acid encoding a Car4 enzyme and/or a Car4 enzyme activator. Certain embodiments of the invention provide the use of a nucleic acid encoding a Car4 enzyme to prepare a medicament useful for treating lung fibrosis and/or emphysema in combination with a Car4 enzyme and/or a Car4 enzyme activator. Certain embodiments of the invention provide the use of a Car4 enzyme activator to prepare a medicament useful for treating lung fibrosis and/or emphysema in combination with a Car4 enzyme and/or a nucleic acid encoding a Car4 enzyme.

Certain embodiments also provide a method of identifying a test agent that is useful for treating lung fibrosis and/or emphysema, the method comprising contacting a Car4 enzyme with a test agent and identifying the test agent as being useful for treating lung fibrosis and/or emphysema when the test agent enhances the expression and/or function of a Car4 enzyme as compared to a control (e.g., a negative control agent or in the absence of an agent).

### BRIEF DESCRIPTION OF THE FIGURES

The following detailed description of exemplary embodiments will be better understood when read in conjunction with the appended Figures. For the purpose of illustration, non-limiting embodiments are shown in the Figures. It should be understood, however, that the instant specification is not limited to the precise arrangements and instrumentalities of the embodiments shown in the Figures.

FIG. 1 illustrates Real-Time PCR evaluation of Car4 in various sort-purified immune cells. P value compared to monocytes (**,p<0.01) Mann-Whitney.

FIG. 2 illustrates expression of Car4 on CD64+F480+CD11C+Siglec-F+alveolar macrophages (AMs) and CD11c+MHCII+dendritic cells (DCs) from naïve lung. The data is representative of three individual experiments.

FIG. 3A-3B illustrates Car4 levels on CD46+F480+CD11C+Siglec-F+AMs from Car4-floxed mice (3A) or Car4-floxed x CD11c-Cre mice (3B). The data is representative of three individual experiments.

FIGS. 4A-4C illustrate (4A, 4B) RT-PCR of naïve lung tissue and (4C) Pulse Ox readings from Car4-floxed and Car4-floxed CD11c-Cre mice. Statistical analysis was performed using a Student's t-test.

FIG. 5 is a heat map illustrating significantly different genes (RNA-seq) between sort-purified Car4-sufficent and -deficient AMs from naïve lung.

FIG. 6 illustrates pathways enriched in Car4−/−AMs as determined by (Ingenuity Pathway Analysis) IPA analysis. Statistical analysis was performed using a Student's t-test (adj. p<0.05).

FIG. 7 illustrates M2-associated genes upregulated in Car4-deficient AMs identified in a type 2 associated pathway by IPA analysis.

FIG. 8 illustrates surface expression of Car4 on AMs from naive or Nippostrongylus brasiliensis (Nb) infected mice (day 7). Statistical analysis was performed using a Students t-test (**, p<0.01).

FIGS. 9A-9C illustrate (9A, 9B), RT-PCR analysis of lung tissue from naïve or Nb infected mice (day 7). Car4-floxed (Car4fl/fl) or Car4-floxed x CD11c-Cre (Car4Cre+) mice. (9C) Pulse Ox analysis of naïve and infected groups of mice. Statistical analysis was performed using a two-way ANOVA (*, p<0.05). (**, p<0.01).

FIGS. 10A-10B illustrate representative lung pathology (trichrome staining) (10A) and FN1 expression in the lungs of Car4fl/fl and Car4fl/fl CD11c-Cre mice on day 7 post-Nb (10B). Statistical analysis was performed using a Student's t-test.

FIG. 11 illustrates expression levels of CA4 and MMP12 from normal lung tissue or Idiopathic Pulmonary Fibrosis (IPF) lung tissue. Wald test with a FDR (benjamini-hochberg) correction.

FIGS. 12A-12C illustrate (12A) Total brochoalveolar lavage (BAL) cells, (12B) surface expression of Car4 on AMs and (12C) and Mmp12 expression in lung tissue on day 14 post-elastase instillation.

FIGS. 13A-13B illustrates evaluation of wild-type (WT) mice instilled with elastase in the lung and treated with either phosphate-buffered saline (PBS) or Car4 enzyme. (13A) peripheral 02 levels were measured on day 7 post-instillation (day 7) and compared to baseline O2 levels (day 0). (13B) Lung pathology was also evaluated (H&E staining).

### DETAILED DESCRIPTION

As described herein, the present invention provides methods for treating lung (or pulmonary) fibrosis and/or emphysema. In certain embodiments, the methods comprise administering to a mammal in need thereof (e.g., such as a human patient) an effective amount of a carbonic anhydrase 4 (Car4) enzyme (e.g., recombinant Car4 enzyme) or a nucleic acid encoding a Car4 enzyme. In certain other embodiments, the methods comprise administering to a mammal in need thereof an effective amount of a Car4 enzyme activator, such as L-Histidine, D-Histidine, L-Phenylalanine, D-Phenylalanine, or a pharmaceutically acceptable salt thereof.

Accordingly, certain embodiments of the invention provide a method of treating lung fibrosis and/or emphysema comprising administering to a mammal in need thereof an effective amount of at least one of a Car4 enzyme, a nucleic acid encoding a Car4 enzyme, or a Car4 enzyme activator.

In certain embodiments, a two- or three-way combination selected from a Car4 enzyme, a nucleic acid encoding a Car4 enzyme, and a Car4 activator, is administered to the mammal. For example, in certain embodiments, a Car4 enzyme and a nucleic acid encoding a Car4 enzyme are administered to the mammal (e.g., the Car4 enzyme may be used to address acute disease aspects while the nucleic acid (e.g., expressed from a vector) may be used to promote long-term benefits). In certain other embodiments, a Car4 enzyme and a Car4 activator are administered. In certain embodiments, a nucleic acid encoding a Car4 enzyme and a Car 4 activator are administered. In certain embodiments, a Car4 enzyme, a nucleic acid encoding a Car4 enzyme, and a Car4 enzyme activator are administered.

When a two- or three-way combination of the above agents are administered, they may be administered either simultaneously or sequentially. In certain embodiments, the agents are administered simultaneously. In certain embodiments, a pharmaceutical composition comprising the two or three agents is administered. Thus, certain embodiments provide a composition (e.g., a pharmaceutical composition) comprising a two- or three-way combination selected from a Car4 enzyme, a nucleic acid encoding a Car4 enzyme, and a Car4 activator. In certain embodiments, the composition further comprises a pharmaceutically acceptable diluent or carrier. In certain embodiments, the agents are administered sequentially. For example, in certain embodiments, the Car4 enzyme is administered first and the nucleic acid encoding the Car4 enzyme is administered second. In certain other embodiments, the nucleic acid encoding the Car4 enzyme is administered first and the Car4 enzyme is administered second. In certain embodiments, the method is a method for the treatment of lung fibrosis.

In certain embodiments, the lung fibrosis and/or emphysema treated with the method described herein is M2 macrophage-mediated. In certain embodiments, the lung fibrosis and/or emphysema is caused by autoimmune diseases (e.g., rheumatoid arthritis, scleroderma, or Sjogren's syndrome), viral infections, gastroesophageal reflux disease (GERD), genetic factors, medications (e.g., drug-induced pulmonary fibrosis), radiation treatments, COVID-19, hypersensitivity pneumonitis, non-specific interstitial pneumonia, and/or pneumoconiosis.

In certain embodiments, the method is for the treatment of lung fibrosis and the lung fibrosis is idiopathic pulmonary fibrosis (IPF).

In certain embodiments, the method is for the treatment of emphysema. In certain embodiments, the mammal has chronic obstructive pulmonary disease (COPD).

In certain embodiments, the lung fibrosis and/or emphysema treated with the method described herein is caused by a parasite infection, such as a helminth infection. In certain embodiments, the parasite infection is an infection caused by a human hookworm, such as Ancylostoma duodenale or Necator americanus.

In certain embodiments, the lung fibrosis and/or emphysema treated with the method described herein is caused by inhalation of noninfectious agents including at least one of a toxin, an irritant, or particulate matter. In certain embodiments, the toxin, irritant or particulate matter is silica dust, asbestos fibers, hard metal dusts, coal dust, grain dust, bird and animal droppings, cigarette smoke, and/or aspirated gastric acid.

In certain embodiments, the method for treating lung fibrosis and/or emphysema comprises intratracheally administering to a mammal in need thereof an effective amount of a Car4 enzyme, a nucleic acid encoding a Car4 enzyme, and/or a Car4 enzyme activator. In certain embodiments, the method for treating lung fibrosis and/or emphysema comprises administering to a mammal in need thereof an effective amount of a Car4 enzyme, a nucleic acid encoding a Car4 enzyme, and/or a Car4 enzyme activator by inhalation or by nebulization. In certain embodiments, the method for treating lung fibrosis and/or emphysema comprises nasally administering to a mammal in need thereof an effective amount of a Car4 enzyme, a nucleic acid encoding a Car4 enzyme, and/or a Car4 enzyme activator. In certain other embodiments, the method for treating lung fibrosis and/or emphysema comprises intravenously administering to a mammal in need thereof an effective amount of a Car4 enzyme, a nucleic acid encoding a Car4 enzyme, and/or a Car4 enzyme activator.

In certain embodiments, the method described herein comprises administering a pharmaceutical composition that comprises a pharmaceutically effective carrier and at least one of a Car4 enzyme, a nucleic acid encoding a Car4 enzyme, or a Car4 enzyme activator.

Certain embodiments of the invention also provide formulations (e.g., pharmaceutical formulations) that include at least one of a Car4 enzyme, a nucleic acid encoding a Car4 enzyme, and/or a Car4 enzyme activator for use in a method described herein.

### Car4 Enzymes

As discussed above, in certain embodiments, a method for treating lung fibrosis and/or emphysema comprises administering to a mammal in need thereof an effective amount of a Car4 enzyme.

The terms “carbonic anhydrase 4 enzyme,” “Car4,” “Car4 enzyme,” “Car4 polypeptide,” and “Car4 amino acid sequence” are used interchangeably and refer to the metabolic enzyme, encoded by the CA4 gene, that catalyzes the conversion between carbon dioxide and bicarbonate (reversible hydration of carbon dioxide: CO₂+H₂O=HCO₃⁻+H⁺), or to its catalytically active fragments and variants. Car4 enzyme, or its active fragments, and variants thereof are known in the art and described herein. In certain embodiments, a Car4 enzyme is a human Car4 enzyme, or an active fragment and/or variant thereof. For example, in certain embodiments, a Car4 enzyme may comprise an amino acid (aa) sequence according to any one of NCBI/UniProt accession numbers P22748 (human), XP_005257696 (human), AAA35625.1 (human), AAA35626.1 (human), 5IPZ_A (human), 1ZNC_A (human), or XP_011523485.1 (human), or active fragments and/or variants thereof (which are incorporated by reference herein). Car4 enzymes have also been characterized in a variety of other mammals. Accordingly, in certain embodiments, a Car4 enzyme may be a non-human, mammalian Car4 enzyme, or an active fragment and/or variant thereof. For example, a Car4 enzyme may comprise an amino acid (aa) sequence derived from, e.g., UniProt Q95323 (bovine), P48284 (rat), Q64444 (mouse), or P48283 (rabbit), or active fragments (e.g., such as Glu18-Ser277 from Q64444) and/or variants thereof (which are incorporated by reference herein). Active site residues of Car4 enzyme, e.g., catalytic residue and zinc-metal binding residues are known in the art and described herein, e.g., T Stams et al., Proc Natl Acad Sci USA. 1996 Nov. 26; 93 (24): 13589-94, DOI: 10.1073/pnas.93.24.13589, which is incorporated by reference herein. In certain embodiments, the Car4 enzyme may be expressed as a full-length preproprotein enzyme that could be further processed into a shorter, mature form or fragment. For example, the signal peptide (e.g., the first 18 aa residues in the N-terminus of full-length enzyme) and/or the pro-peptide (e.g., the last 28 aa residues in the C-terminal of full-length enzyme) may be removed in its mature form (e.g., see UniProt accession number P22748 or SEQ ID NO: 1 below for a full length Car4 enzyme sequence; see, e.g., SEQ ID NO:3 as an example of sequence for a mature form/fragment). The term “Car4 enzyme” also includes active fragments and/or variants that have, including but not limited to, one or more amino acid substitution(s), and/or N-terminal and/or C-terminal truncation or addition, such as tag (e.g., purification or affinity tag such as His6 tag), as compared to the full-length preproprotein, as long as the active fragment or variant is capable of catalyzing the conversion between carbon dioxide and bicarbonate (reversible hydration of carbon dioxide: CO₂+H₂O=HCO₃⁻+H⁺). As used herein, the term “recombinant Car4 enzyme” refers to a recombinantly constructed or produced Car4 enzyme, active fragment, or variant, which may optionally lack the N-terminal signal peptide and/or the C-terminal pro-peptide.

In certain embodiments, the Car4 enzyme is a wild-type Car4 enzyme (e.g., a wild-type human Car4 enzyme). In some embodiments, the Car4 enzyme is a variant or a catalytically active fragment of a Car4 polypeptide or protein (e.g., comprises a Car4 amino acid sequence described herein). In certain embodiments, the Car4 enzyme is a Car4 enzyme variant (as compared to a wild-type Car4 enzyme). In certain embodiments, the Car4 enzyme is a catalytically active fragment of a Car4 enzyme or a Car4 enzyme variant. In some embodiments, a catalytically active variant or fragment of a Car4 enzyme has at least 50%, at least 55%, at least 60%, at least 65%, at least 70%, at least 75%, at least 80%, at least 85%, at least 90%, at least 95%, or greater of the activity of the wild-type Car4 enzyme.

In certain embodiments, the Car4 enzyme is about 222 to 336, 246 to 312, 266 to 312, or 266 to 294 amino acids (aa) in length. In certain embodiments, the Car4 enzyme is about 266 to 312 aa in length. In certain embodiments, the Car4 enzyme is about 312 aa in length. In certain embodiments, the Car4 enzyme is about 266 aa in length. In certain embodiments, the Car4 enzyme is about 294 aa in length. In certain embodiments, the Car4 enzyme is about 267 aa in length. In certain embodiments, the Car4 enzyme is about 305 aa in length. In certain embodiments, the Car4 enzyme is about 260 aa in length. In certain embodiments, the Car4 enzyme is about 246 aa in length. In certain embodiments, the Car4 enzyme is about 222 aa in length.

In certain embodiments, the Car4 enzyme comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:1, 2, or 3.

(SEQ ID NO: 1)
MRMLLALLALSAARPSASAESHWCYEVQAESSNYPCLVPVKWGGNCQKDR
QSPINIVTTKAKVDKKLGRFFFSGYDKKQTWTVQNNGHSVMMLLENKASI
SGGGLPAPYQAKQLHLHWSDLPYKGSEHSLDGEHFAMEMHIVHEKEKGTS
RNVKEAQDPEDEIAVLAFLVEAGTQVNEGFQPLVEALSNIPKPEMSTTMA
ESSLLDLLPKEEKLRHYFRYLGSLTTPTCDEKVVWTVFREPIQLHREQIL
AFSQKLYYDKEQTVSMKDNVRPLQQLGORTVIKSGAPGRPLPWALPALLG
PMLACLLAGFLR,
(SEQ ID NO: 2)
MRMLLALLALSAARPSASAESHWCYEVQAESSNYPCLVPVKWGGNCQKDR
QSPINIVTTKAKVDKKLGRFFFSGYDKKQTWTVQNNGHSVMMLLENKASI
SGGGLPAPYQAKQLHLHWSDLPYKGSEHSLDGEHFAMEMHIVHEKEKGTS
RNVKEAQDPEDEIAVLAFLVEIGRMNWPPPLAPCRLSQDPSLPFQAGTQV
NEGFQPLVEALSNIPKPEMSTTMAESSLLDLLPKEEKLRHYFRYLGSLTT
PTCDEKVVWTVFREPIQLHREQILAFSQKLYYDKEQTVSMKDNVRPLQQL
GQRTVIKSGAPGRPLPWALPALLGPMLACLLAGFLR,
(SEQ ID NO: 3)
AESHWCYEVQAESSNYPCLVPVKWGGNCQKDRQSPINIVTTKAKVDKKLG
RFFFSGYDKKQTWTVQNNGHSVMMLLENKASISGGGLPAPYQAKQLHLHW
SDLPYKGSEHSLDGEHFAMEMHIVHEKEKGTSRNVKEAQDPEDEIAVLAF
LVEAGTQVNEGFQPLVEALSNIPKPEMSTTMAESSLLDLLPKEEKLRHYF
RYLGSLTTPTCDEKVVWTVFREPIQLHREQILAFSQKLYYDKEQTVSMKD
NVRPLQQLGQRTVIKS

In certain embodiments, the Car4 enzyme comprises an amino acid sequence having at least about 80% sequence identity to any one of SEQ ID NOs: 1 to 3.

In certain embodiments, the Car4 enzyme comprises an amino acid sequence having at least about 80% sequence identity to SEQ ID NO:1. In certain embodiments, the Car4 enzyme comprises an amino acid sequence having at least about 86%, 87%, 88%, 89%, 90%, 91%, 92%, 93%, 94%, 95%, 96%, 97%, 98%, 99%, or 100% identity to SEQ ID NO:1. In certain embodiments, the Car4 enzyme comprises an amino acid sequence having at least about 90% sequence identity to SEQ ID NO:1. In certain embodiments, the Car4 enzyme comprises an amino acid sequence having at least about 95% sequence identity to SEQ ID NO:1. In certain embodiments, the Car4 enzyme comprises SEQ ID NO:1. In certain embodiments, the Car4 enzyme consists of SEQ ID NO:1.

In certain embodiments, the Car4 enzyme comprises an amino acid sequence having at least about 80% sequence identity to SEQ ID NO:2. In certain embodiments, the Car4 enzyme comprises an amino acid sequence having at least about 86%, 87%, 88%, 89%, 90%, 91%, 92%, 93%, 94%, 95%, 96%, 97%, 98%, 99%, or 100% identity to SEQ ID NO:2. In certain embodiments, the Car4 enzyme comprises an amino acid sequence having at least about 90% sequence identity to SEQ ID NO:2. In certain embodiments, the Car4 enzyme comprises an amino acid sequence having at least about 95% sequence identity to SEQ ID NO:2. In certain embodiments, the Car4 enzyme comprises SEQ ID NO:2. In certain embodiments, the Car4 enzyme consists of SEQ ID NO:2.

In certain embodiments, the Car4 enzyme comprises an amino acid sequence having at least about 80% sequence identity to SEQ ID NO:3. In certain embodiments, the Car4 enzyme comprises an amino acid sequence having at least about 86%, 87%, 88%, 89%, 90%, 91%, 92%, 93%, 94%, 95%, 96%, 97%, 98%, 99%, or 100% identity to SEQ ID NO:3. In certain embodiments, the Car4 enzyme comprises an amino acid sequence having at least about 90% sequence identity to SEQ ID NO:3. In certain embodiments, the Car4 enzyme comprises an amino acid sequence having at least about 95% sequence identity to SEQ ID NO:3. In certain embodiments, the Car4 enzyme comprises SEQ ID NO:3. In certain embodiments, the Car4 enzyme consists of SEQ ID NO:3.

In certain embodiments, an enzyme polypeptide as described herein is fused to a tag (e.g., affinity tag and/or detectable tag such as HIS tag, FLAG tag, or C-Myc tag) via an optional linker sequence, such as Glycine linker or Glycine-serine linker (e.g., GGGG, or GGGS, etc.).

In certain embodiments, the enzyme polypeptide as described herein is present in a fusion polypeptide or fusion protein. For example, the enzyme polypeptide may be joined either directly (i.e., through a peptide bond) or through a peptide/polypeptide linker to another polypeptide (e.g., a polypeptide tag or an antibody fragment, such as an immunoglobulin Fc domain). For example, in certain embodiments, the Car4 enzyme is linked either directly or through a peptide/polypeptide linker to a Fc domain, or fragment thereof (e.g., IgG1, IgG2, IgG3, or IgG4 Fc domain, or fragment thereof).

### Nucleic Acids Encoding a Car 4 Enzyme

As discussed above, in certain embodiments, a method for treating lung fibrosis and/or emphysema comprises administering to a mammal in need thereof an effective amount of a nucleic acid encoding a Car4 enzyme.

Nucleic acids encoding a Car4 enzyme, or its active fragments, and variants are known in the art and described herein. For example, in certain embodiments, a nucleic acid encoding a Car4 enzyme, or its active fragments, and variants may comprise a nucleic acid sequence according to any one of NCBI accession number NM_000717.5, XM_047436653.1, XM_047436656.1, XM_005257639.4, XM_047436652.1, which are incorporated by reference herein.

In certain embodiments, the nucleic acid encodes a Car4 enzyme polypeptide described herein. For example, in certain embodiments, the nucleic acid encodes a Car4 enzyme polypeptide comprising an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:1, 2, or 3.

In certain embodiments, the nucleic acid is DNA.

In certain embodiments, the nucleic acid is RNA, for example, mRNA.

In certain embodiments, the nucleic acid encoding a Car4 enzyme comprises a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 4 or SEQ ID NO:5.

(SEQ ID NO: 4)
ACCCGCGGCGGCCTCCTCGGTGCGCGACCCCCGGCTCAGAGGACTCTTTG
CTGTCCCGCAAGATGCGGATGCTGCTGGCGCTCCTGGCCCTCTCCGCGGC
GCGGCCATCGGCCAGTGCAGAGTCACACTGGTGCTACGAGGTTCAAGCCG
AGTCCTCCAACTACCCCTGCTTGGTGCCAGTCAAGTGGGGTGGAAACTGC
CAGAAGGACCGCCAGTCCCCCATCAACATCGTCACCACCAAGGCAAAGGT
GGACAAAAAACTGGGACGCTTCTTCTTCTCTGGCTACGATAAGAAGCAAA
CGTGGACTGTCCAAAATAACGGGCACTCAGTGATGATGTTGCTGGAGAAC
AAGGCCAGCATTTCTGGAGGAGGACTGCCTGCCCCATACCAGGCCAAACA
GTTGCACCTGCACTGGTCCGACTTGCCATATAAGGGCTCGGAGCACAGCC
TCGATGGGGAGCACTTTGCCATGGAGATGCACATAGTACATGAGAAAGAG
AAGGGGACATCGAGGAATGTGAAAGAGGCCCAGGACCCTGAAGACGAAAT
TGCGGTGCTGGCCTTTCTGGTGGAGGCTGGAACCCAGGTGAACGAGGGCT
TCCAGCCACTGGTGGAGGCACTGTCTAATATCCCCAAACCTGAGATGAGC
ACTACGATGGCAGAGAGCAGCCTGTTGGACCTGCTCCCCAAGGAGGAGAA
ACTGAGGCACTACTTCCGCTACCTGGGCTCACTCACCACACCGACCTGCG
ATGAGAAGGTCGTCTGGACTGTGTTCCGGGAGCCCATTCAGCTTCACAGA
GAACAGATCCTGGCATTCTCTCAGAAGCTGTACTACGACAAGGAACAGAC
AGTGAGCATGAAGGACAATGTCAGGCCCCTGCAGCAGCTGGGGCAGCGCA
CGGTGATAAAGTCCGGGGCCCCGGGTCGGCCGCTGCCCTGGGCCCTGCCT
GCCCTGCTGGGCCCCATGCTGGCCTGCCTGCTGGCCGGCTTCCTGCGATG
ATGGCTCACTTCTGCACGCAGCCTCTCTGTTGCCTCAGCTCTCCAAGTTC
CAGGCTTCCGGTCCTTAGCCTTCCCAGGTGGGACTTTAGGCATGATTAAA
ATATGGACATATTTTTGGAGAAA
(SEQ ID NO: 5)
GCAGAGTCACACTGGTGCTACGAGGTTCAAGCCGAGTCCTCCAACTACCC
CTGCTTGGTGCCAGTCAAGTGGGGTGGAAACTGCCAGAAGGACCGCCAGT
CCCCCATCAACATCGTCACCACCAAGGCAAAGGTGGACAAAAAACTGGGA
CGCTTCTTCTTCTCTGGCTACGATAAGAAGCAAACGTGGACTGTCCAAAA
TAACGGGCACTCAGTGATGATGTTGCTGGAGAACAAGGCCAGCATTTCTG
GAGGAGGACTGCCTGCCCCATACCAGGCCAAACAGTTGCACCTGCACTGG
TCCGACTTGCCATATAAGGGCTCGGAGCACAGCCTCGATGGGGAGCACTT
TGCCATGGAGATGCACATAGTACATGAGAAAGAGAAGGGGACATCGAGGA
ATGTGAAAGAGGCCCAGGACCCTGAAGACGAAATTGCGGTGCTGGCCTTT
CTGGTGGAGGCTGGAACCCAGGTGAACGAGGGCTTCCAGCCACTGGTGGA
GGCACTGTCTAATATCCCCAAACCTGAGATGAGCACTACGATGGCAGAGA
GCAGCCTGTTGGACCTGCTCCCCAAGGAGGAGAAACTGAGGCACTACTTC
CGCTACCTGGGCTCACTCACCACACCGACCTGCGATGAGAAGGTCGTCTG
GACTGTGTTCCGGGAGCCCATTCAGCTTCACAGAGAACAGATCCTGGCAT
TCTCTCAGAAGCTGTACTACGACAAGGAACAGACAGTGAGCATGAAGGAC
AATGTCAGGCCCCTGCAGCAGCTGGGGCAGCGCACGGTGATAAAGTCC

In certain embodiments, the nucleic acid encoding a Car4 enzyme comprises a nucleic acid sequence having at least 80% sequence identity to SEQ ID NO: 4. In certain embodiments, the nucleic acid encoding a Car4 enzyme comprises a nucleic acid sequence having at least about 90% sequence identity to SEQ ID NO:4. In certain embodiments, the nucleic acid encoding a Car4 enzyme comprises a nucleic acid sequence having at least about 95% sequence identity to SEQ ID NO:4. In certain embodiments, the nucleic acid encoding a Car4 enzyme comprises SEQ ID NO:4. In certain embodiments, the nucleic acid encoding a Car4 enzyme consists of SEQ ID NO:4.

In certain embodiments, the nucleic acid encoding a Car4 enzyme comprises a nucleic acid sequence having at least 80% sequence identity to SEQ ID NO: 5. In certain embodiments, the nucleic acid encoding a Car4 enzyme comprises a nucleic acid sequence having at least about 90% sequence identity to SEQ ID NO:5. In certain embodiments, the nucleic acid encoding a Car4 enzyme comprises a nucleic acid sequence having at least about 95% sequence identity to SEQ ID NO:5. In certain embodiments, the nucleic acid encoding a Car4 enzyme comprises SEQ ID NO:5. In certain embodiments, the nucleic acid encoding a Car4 enzyme consists of SEQ ID NO:5.

In certain embodiments, the nucleic acid is comprised within an expression cassette, wherein the nucleic acid is operably linked to a promoter.

In certain embodiments, the nucleic acid encoding the Car4 enzyme or expression cassette is comprised within a vector. Any suitable vector may be used for introducing a nucleic acid/expression cassette described herein into a mammalian cell. Examples of suitable vectors include plasmids, cosmids, phage, liposomes, molecular conjugates, and viruses. In certain embodiments, the vector is a plasmid. In certain embodiments, the vector is a viral vector, for example, an adeno-associated virus vector (AAV).

Thus, in certain embodiments, a method described herein comprises administering an effective amount of an expression cassette or vector (e.g., a viral vector, such as AAV viral particles) comprising the nucleic acid encoding the Car4 enzyme to the mammal.

In certain embodiments, the nucleic acid (e.g., mRNA) is comprised within a liposome or nanoparticle (e.g., lipid nanoparticle). Thus, in certain embodiments, a method described herein comprises administering an effective amount of a liposome or nanoparticle comprising the nucleic acid encoding the Car4 enzyme to the mammal.

### Car4 Enzyme Activators

As discussed above, in certain embodiments, a method for treating lung fibrosis and/or emphysema comprises administering to a mammal in need thereof an effective amount of a Car4 enzyme activator.

As used herein, the term “carbonic anhydrase 4 enzyme activator (Car4 enzyme activator)” includes any therapeutic agent or compound or treatment capable of enhancing the expression and/or function of a Car4 enzyme (e.g., enhances transcription, RNA maturation, RNA translation, post-translational modification, or enzymatic activity (e.g., enhances activation of the enzyme)). For example, in certain embodiments, the activator detectably enhances the expression level or enzymatic activity of a Car4 enzyme as measured, e.g., using an assay described herein. In certain embodiments, the activator enhances the expression level or enzymatic activity of a Car4 enzyme by at least about 5%, at least about 10%, at least about 20%, at least about 30%, at least about 40%, at least about 50%, at least about 60%, at least about 70%, at least about 80%, or at least about 90%.

The activator may be of natural or synthetic origin. For example, it may be a polypeptide, a protein, a peptide, or small molecule (e.g., an organic compound).

The term “small molecule” includes organic molecules having a molecular weight of less than about 1000 amu. In one embodiment a small molecule can have a molecular weight of less than about 800 amu. In another embodiment a small molecule can have a molecular weight of less than about 500 amu.

In certain embodiments, the activator is a selective Car4 activator. For example, the Car4 activator may be at least 5, at least 10, at least 50, at least 100, at least 500, or at least 1,000 fold selective for Car4 over another Car in a selected assay.

Non-limiting examples of Car4 enzyme activators that may be used in the present invention include L-phenylalanine, D-phenylalanine, L-histidine, D-histidine, or a pharmaceutically acceptable salt thereof. Thus, in certain embodiments, the Car4 enzyme activator comprises at least one of L-phenylalanine, D-phenylalanine, L-histidine, D-histidine, or a pharmaceutically acceptable salt thereof. In certain embodiments, a combination of Car4 enzyme activators are administered.

### Additional Therapeutic Agents

In certain embodiments, a method for treating lung fibrosis and/or emphysema as described herein further comprises administering one or more additional therapeutic agents or therapies to the mammal.

In certain embodiments, the one or more additional therapeutic agents or therapies are useful for treating lung fibrosis (e.g., Nintebanib or Pirfenidone). In certain embodiments, the one or more additional therapeutic agents or therapies is Nintedanib. In certain embodiments, the one or more additional therapeutic agents is Pirfenidone.

In certain embodiments, the one or more additional therapeutic agent or therapies are useful for treating emphysema.

In certain embodiments, the one or more additional therapeutic agents or therapies is an anti-inflammatory agent. In certain embodiments, the one or more additional therapeutic agents or therapies is a corticosteroid, an anticholinergic, and/or a beta2-adrenergic agonist.

In certain embodiments, the one or more additional therapeutic agents or therapies may be administered either simultaneously or sequentially with a Car4 enzyme, a nucleic acid encoding a Car4 enzyme, and/or a Car4 enzyme activator. In certain embodiments, the one or more additional therapeutic agents is administered simultaneously with the Car4 enzyme, nucleic acid encoding Car4 enzyme, and/or Car4 enzyme activator. In certain embodiments, a pharmaceutical composition comprising the Car4 enzyme, nucleic acid encoding Car4 enzyme, and/or Car4 enzyme activator and the at least one other therapeutic agent is administered. In certain embodiments, the Car4 enzyme, nucleic acid encoding Car4 enzyme, and/or Car4 enzyme activator and the one or more additional therapeutic agents are administered sequentially. In certain embodiments, the Car4 enzyme, nucleic acid encoding Car4 enzyme, and/or Car4 enzyme activator is administered first and one or more additional therapeutic agents is administered second. In certain embodiments, the one or more additional therapeutic agents is administered first and the Car4 enzyme, nucleic acid encoding Car4 enzyme, and/or Car4 enzyme activator is administered second.

### Administration

Compounds and agents described herein (e.g., Car4 enzymes, Car4 enzyme activators, nucleic acids encoding Car4 enzymes, and additional therapeutic agents) can be formulated as pharmaceutical compositions and administered to a mammalian host, such as a human patient in a variety of forms adapted to the chosen route of administration, e.g., orally or parenterally, by intravenous, intramuscular, topical, sub-lingual, optical (e.g., eye drops), nasal (i.e., nasal spray) or subcutaneous routes. In certain embodiments, the compounds/agents may be delivered via delayed administration. In certain embodiments, the compounds/agents may be administered via nasal or pulmonary delivery, for example, via intratracheal delivery or via inhalation through an inhaler or nebulizer. Delivery of protein or nucleic acids to the lung, or pulmonary delivery of protein or nucleic acids are known in the art and described herein, for example, U.S. Pat. Nos. 6,737,045; 8,007,780; 9,597,413; and 9,725,522 are incorporated by reference herein.

Thus, the present compounds/agents may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound/agent may be combined with one or more excipients and used in the form of ingestible tablets, buccal tablets, troches, capsules, elixirs, suspensions, syrups, wafers, and the like. Such compositions and preparations should contain at least 0.1% of active compound/agent. The percentage of the compositions and preparations may, of course, be varied and may conveniently be between about 2 to about 60% of the weight of a given unit dosage form. The amount of active compound/agent in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound/agent, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound/agent may be incorporated into sustained-release preparations and devices.

The active compound/agent may also be administered intravenously or intraperitoneally by infusion or injection. Solutions of the active compound/agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound/agent in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds/agents may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the compounds/agents can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds/agents (e.g., Car4 enzyme, Car4 enzyme activator, or nucleic acid encoding a Car4 enzyme) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound/agent, or an active salt or derivative thereof, required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator.

As described herein, the Car4 enzymes, Car4 enzyme activators, and/or nucleic acids encoding a Car4 enzyme can also be administered in combination with other therapeutic agents, for example, other therapeutic agents described herein. Accordingly, in one embodiment the invention also provides a composition comprising 1) a Car4 enzyme, Car4 enzyme activator, and/or nucleic acid encoding a Car4 enzyme, 2) at least one other therapeutic agent, and 3) a pharmaceutically acceptable diluent or carrier. The invention also provides a kit comprising 1) a Car4 enzyme, Car4 enzyme activator, and/or nucleic acid encoding a Car4 enzyme, 2) at least one other therapeutic agent, 3) packaging material, and 4) instructions for administering the Car4 enzyme, Car4 enzyme activator, and/or nucleic acid encoding a Car4 enzyme and the other therapeutic agent or agents to a mammal to treat lung fibrosis and/or emphysema.

### Certain Definitions

The terms “peptide”, “polypeptide” and “protein” are used interchangeably herein. Polypeptide sequences specifically recited herein are written with the amino terminus on the left and the carboxy terminus on the right. A protein molecule (e.g., enzyme) may exist in an isolated or purified form, for example, an isolated or purified form as an active ingredient of a drug dosage form. Fragments and variants of the disclosed proteins or partial-length proteins encoded thereby are also encompassed by the present invention.

The invention encompasses isolated or substantially purified protein compositions. In the context of the present invention, an “isolated” or “purified” polypeptide is a polypeptide that exists apart from its native environment. A polypeptide may exist in a purified form or may exist in a non-native environment such as, for example, a transgenic host cell such as a bacterium or a mammalian expression system for the production of the polypeptide. For example, an “isolated” or “purified” protein, or biologically active fragment thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active fragment thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention.

In certain embodiments, one or more amino acid residues are mutated within the polypeptide as described herein. For example, the mutation is conducted via error-prone PCR or site directed mutagenesis. In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10:6487-6500 (1982); Wang A. et al., Science 224:1431-1433; Dalbadie-McFarland G. et al., Proc. Natl. Acad. Sci. U.S.A. 79:6409-6413 (1982)). The number of mutated amino acids is not limited, but in general, the number falls within 40% of amino acids, and specifically within 35%, and still more specifically within 30% (e.g., within 25%). The identity of amino acid sequences can be determined as described herein. In certain embodiments, one or more amino acid residue is mutated into one that is a non-conservative substitution.

The polypeptides obtained can be purified to homogeneity. The polypeptides can be isolated and purified by a method routinely used to isolate and purify proteins. The polypeptides can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography (e.g., metal affinity chromatography), ion exchange chromatography, hydrophobic chromatography, gel filtration, reverse-phase chromatography, and adsorption chromatography. These chromatographic methods can be practiced using liquid phase chromatography, such as HPLC and FPLC. The polypeptides can also be purified by utilizing target binding, using carriers on which targets have been immobilized.

The polypeptides of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the polypeptides of the invention, and pharmaceutically acceptable carriers and/or additives. Other exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “comparison window” makes reference to a contiguous and specified segment of an amino acid or polynucleotide sequence, wherein the sequence in the comparison window may comprise additions or deletions (i.e., gaps) compared to the reference sequence (which does not comprise additions or deletions) for optimal alignment of the two sequences. Generally, the comparison window is at least about 20 contiguous amino acid residues or nucleotides in length, and optionally can be 30, 40, 50, 100, or longer.

As used herein, “percentage of sequence identity” means the value determined by comparing two optimally aligned sequences over a comparison window, wherein the portion of the polypeptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least about 70%, 71%, 72%, 73%, 74%, 75%, 76%, 77%, 78%, or 79%, at least about 80%, 81%, 82%, 83%, 84%, 85%, 86%, 87%, 88%, or 89%, at least about 90%, 91%, 92%, 93%, or 94%, and at least about 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least about 70%, at least about 80%, 90%, or at least about 95%.

The term “substantial identity” in the context of a peptide indicates that a peptide comprises a sequence with at least about 70%, 71%, 72%, 73%, 74%, 75%, 76%, 77%, 78%, or 79%, 80%, 81%, 82%, 83%, 84%, 85%, 86%, 87%, 88%, or 89%, at least about 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity or complementarity for the test sequence(s) relative to the reference sequence, based on the designated program parameters.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., dehydroalanine, homoserine, phosphoserine, phosphothreonine, phosphotyrosine, hydroxyproline, gamma-carboxyglutamate; hippuric acid, octahydroindole-2-carboxylic acid, statine, 1,2,3,4, -tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein) The term also comprises natural and unnatural amino acids bearing a cyclopropyl side chain or an ethyl side chain.

The term “nucleic acid” and “polynucleotide” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

A “vector” is defined to include, inter alia, any plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position+1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

As used herein, the term “operably linked” refers to a linkage of two elements in a functional relationship. For example, “operably linked” may refer to a linkage of polynucleotide (or polypeptide) elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association of two chemical moieties so that the function of one is affected by the other, e.g., an arrangement of elements wherein the components so described are configured so as to perform their usual function.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

The term “effective amount” or “therapeutically effective amount,” in reference to treating a disease state/condition, refers to an amount of a polypeptide either alone or as contained in a pharmaceutical composition that produces therapeutic effect or is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder, such as lung fibrosis and/or emphysema. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the terms “for use in combination” may refer to the sequential or simultaneous use of specified agents, wherein the agents may be used separately or in a single composition for simultaneous use.

As used herein, the term “therapeutic agent” refers to any agent or material that has a beneficial effect on the mammalian recipient.

The term “mammal” refers to any mammalian species such as a human, mouse, rat, dog, cat, hamster, guinea pig, rabbit, livestock, and the like. Accordingly, in certain embodiments, the mammal is a human, mouse, rat, dog, cat, hamster, guinea pig, rabbit or livestock. In certain embodiments, the mammal is a patient (e.g., a human patient). In certain embodiments, the mammal is a pet, such a dog, cat, hamster, guinea pig or rabbit. In certain embodiments, the mammal is a livestock mammal (e.g., a cow, sheep, horse, pig, chicken, etc.).

“Wild-type” refers to the normal gene, or organism found in nature without any known mutation.

The invention will now be illustrated by the following non-limiting Example.

### Example 1

### Introduction

Recent studies have identified that the metabolic enzyme carbonic anhydrase 4 (Car4) is a lineage identifying marker of alveolar macrophages^{8,9}. These data provoke the hypothesis that Car4 and/or Car4 activators may play important roles in regulating the biologic functions of AMs. Therefore, a novel Car4-floxed mouse was generated and, for the first time, Car4 in alveolar macrophages (Car4-AM^{-/-}) were deleted. Remarkably, Car4-AM^{-/-} mice exhibited elevated expression of il17 and il13 in the lung, showed indications of spontaneous fibrosis and had significantly lower baseline O2 levels. Further, RNA-seq studies revealed that Car4-deficient AMs exhibit a spontaneous M2 macrophage and profibrotic phenotype compared to control AMs. Collectively, these studies demonstrate that Car4 plays a critically important role in regulating the homeostatic functions of AMs.

Few, if any, areas of research have demonstrated more about the plasticity and varied functions of macrophage populations than those investigating host responses to helminth parasites¹⁰⁻¹³. The prevalence of soil-transmitted helminth (STH) infections is estimated to be 2 billion individuals worldwide with an additional 870 million children at risk of becoming infected. Infected individuals can suffer from malnutrition, growth retardation, impaired cognitive function, anemia and severe tissue fibrosis^{14,15} Protective immunity to helminth parasites is dependent on the induction of acute type 2 immune responses that promote the development of M2 macrophages needed to support worm expulsion and mitigate tissue injury that ensues as the parasites migrate through vital organs, such as the lung, as part of their natural life cycle¹⁶. It should be noted that the lung is increasingly recognized as a significant checkpoint for development of protective immunity during helminth infection¹⁷⁻¹⁹. Type 2 immunity thus simultaneously promotes both resistance (parasite killing) and tolerance mechanisms (wound healing) with strong evidence indicating that AMs are critical regulators of both of these host-protective responses^{15, 19, 20}.

As stated above, the data presented herein demonstrate that Car4-deficient AMs have a spontaneous M2 phenotype. Given that alternatively activated AMs play such important roles in promoting protective immunity to parasitic helminths^{10, 13}, an evaluation of whether Car4 expression by AMs is altered following infection with the hookworm Nippostrongylus brasiliensis (Nb) was undertaken. Interestingly, Car4 expression was found to be significantly increased on the surface of AMs post-Nb, suggesting that Car4 may play a role in regulating antihelminth responses in the lung. To further evaluate this possibility, wild-type (WT) and Car4-AM^{-/-} mice were infected with Nb and monitored for infection-induced lung inflammation. Importantly, Car4-AM^{-/-} mice exhibited enhanced expression of M2- and fibrosis-associated markers and suffered from significantly reduced O₂ levels. These data demonstrate that Car4 restricts AM activation to prevent excessive tissue remodeling/fibrosis following the induction of acute inflammation.

The Car4-AM^{-/-} model described herein presented with features of idiopathic lung inflammation and fibrosis. Therefore, an investigation of whether a reduction of human Carbonic anhydrase 4 (CA4) is a feature of human IPF was performed. Importantly, CA4 was one of the most downregulated genes in the lungs of human IPF patients. Additionally, human IPF lungs also exhibit elevated levels of serval genes that are highly upregulated in the present Car4-AM^{-/-} model, suggesting that deletion of Car4 on AMs promotes characteristics of human disease. This work strongly suggests that Car4 is an unappreciated therapeutic target for the treatment of lung fibrosis. To directly test this, a mouse model of elastase-induced lung fibrosis was employed and evaluated to determine whether exogenous Car4 treatment was sufficient to alter parameters of diseases. Importantly, treatment with exogenous Car4 alleviated elastase-induced reductions in oxygen and lung pathology. Collectively, these studies identify Car4 as a previously unappreciated therapeutic target for the treatment of pathologic pulmonary inflammation and fibrosis.

### Results

### Carbonic Anhydrase 4 is the Signature Gene Expressed by AMs

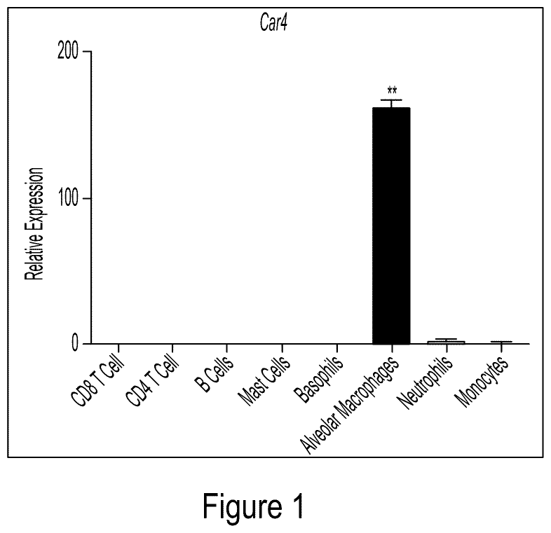

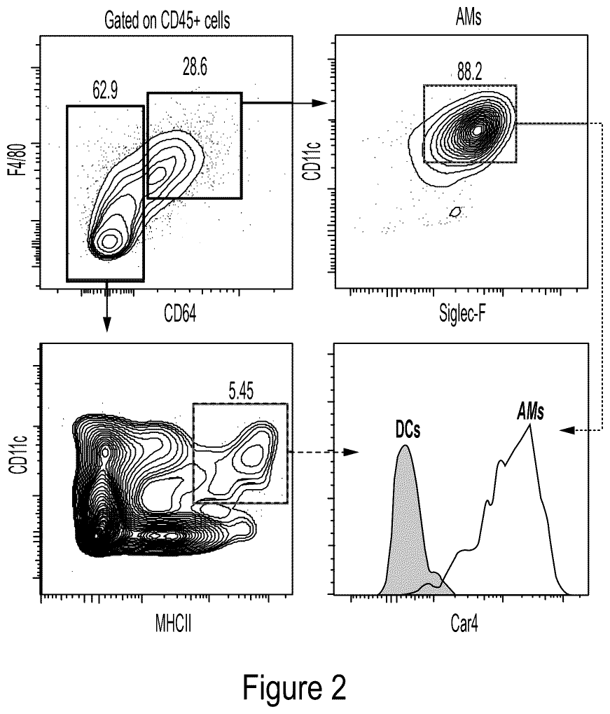

Recent studies indicate that the tissue microenvironment plays a critical role in shaping the phenotype and function of macrophage populations^{21,22}. The data presented herein, as well as other recent data, demonstrate that monocyte-derived macrophages take on characteristics of tissue-derived macrophages after entering the tissue environment^{8,23}. Carbonic anhydrases are a family of enzymes that are known to regulate CO₂ and pH homeostasis, but recent studies suggest that Car enzymes also play important roles in regulating immune cell development and activation²⁴⁻²⁷. For example, it has been demonstrated that Carl is highly expressed by mast cell progenitors and is necessary for mast cell lineage commitment in both mice and humans²⁶⁻²⁸. Further, it was recently demonstrated that Car6b operates as part of an epigenetic complex that regulates cytokine production in macrophages. These data suggest that specific Car enzyme family members may regulate important biological processes in distinct cell lineages. Although it was demonstrated that Car4 was the signature gene expressed by AMs8, its expression profiles in other hematopoietic lineages remain unknown. Therefore, an investigation was undertaken to determine whether Car4 expression is unique to AMs or is shared across other immune cells. To evaluate this, various immune cell populations from naïve mice were sort-purified and their expression of Car4 was determined. Car4 expression was found to be ~150-fold higher in AMs than in T cells, B cells, various granulocytes and monocytes (FIG. 1). Next, a flow cytometric staining protocol was developed to evaluate Car4 at the protein levels. Consistent with transcriptional data, Car4 was robustly expressed on the surface of AMs but was not expressed by other cell lineages including dendritic cells (DCs) (FIG. 2). These data demonstrate that within the immune compartment, Car4 is uniquely expressed by AMs.

### Deletion of Car4 in AMs Results in Altered Function

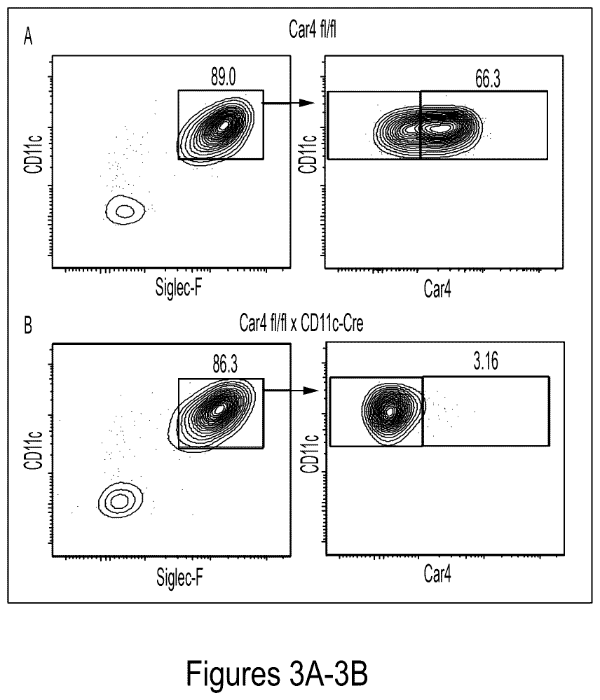

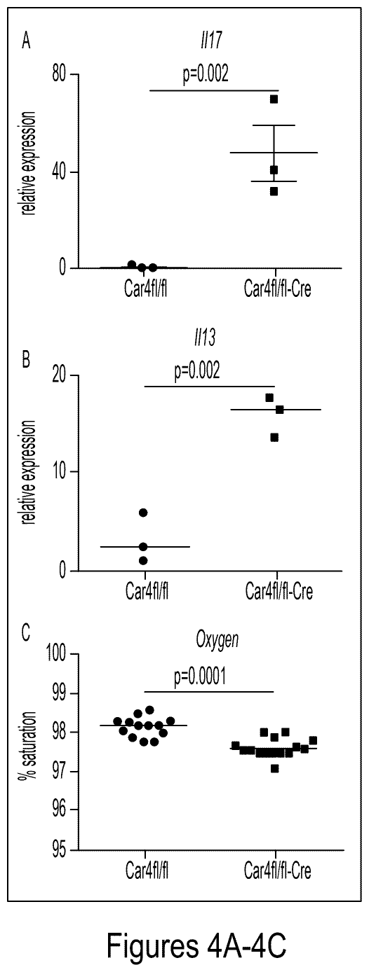

Data presented herein, and that of others, has identified that Car4 expression is a lineage identifying feature of AMs (FIG. 2,3)^{8,9}. Despite these advances, the role of Car4 in regulating AM function remains to be defined. To further investigate the lineage-specific roles of Car4, a novel Car4-floxed mouse model was developed to achieve specific deletions of Car4 using lineages-specific Cre expression systems. As a targeting strategy, CRISPR-Cas9 was used to insert loxP sites in front of exon 3 and behind exon 7 of Car4. To validate this approach, Car4-floxed mice were bred to CD11c-Cre mice that target AMs and DCs^{29,30}. Given that DCs were found to lack expression of Car4 (FIG. 2), this mouse model is largely AM-specific and will be referred to herein as Car4-AM^{-/-}. Flow cytometric analysis confirmed the successful deletion of Car4 in AMs (FIG. 3). Interestingly, Car4-AM^{-/-} mice exhibited increased baseline levels of Il17 and Il13 expression in the lung compared to controls (FIG. 4A,B). Importantly, IL-17 and IL-13 are cytokines known to promote lung fibrosis³¹⁻³⁵. Consistent with spontaneous pulmonary inflammation, Car4-AM^{-/-} mice also presented with significantly decreased O2 levels (FIG. 4C). It is well established that AMs are highly specialized cells that play important homeostatic functions in the lung^{5,6}. The data presented herein demonstrate that Car4 is required for AMs to prevent spontaneous inflammation in the lung.

### Deletion of Car4 in AMs Results in a Dramatically Altered Cellular Phenotype and the Induction of Profibrotic Pathways

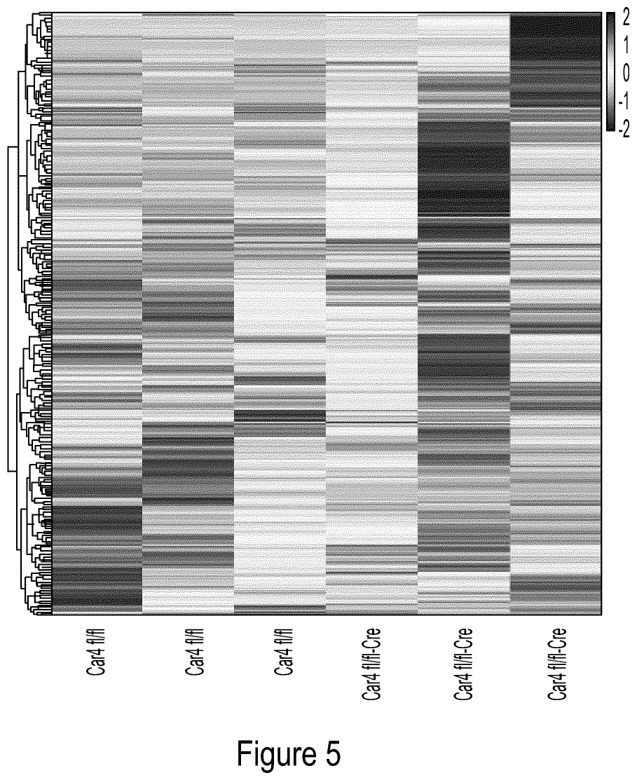

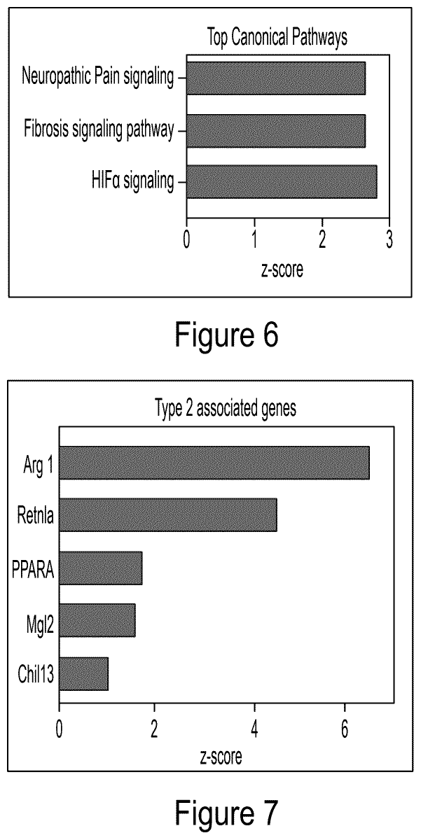

The data presented herein demonstrate that Car4 is a lineage identifying gene of AMs (FIG. 1,2). Further, the data show that deletion of Car4 in AMs is sufficient to promote spontaneous or idiopathic inflammation that is associated with reduced lung function (FIG. 4). Therefore, further investigation was undertaken to determine how a deficiency in Car4 may alter AM functions in a manner that promotes idiopathic inflammation. To test this, AMs from Car4-floxed mice and Car4-AM^{-/-} mice were sort-purified and RNA-seq analysis was performed. Importantly, AMs from Car4-AM^{-/-} mice had 592 significantly upregulated genes compared to control AMs (FIG. 5). Ingenuity Pathway Analysis (IPA) revealed that Car4-deficient AMs were enriched for genes associated with tissue fibrosis, hypoxia inducible factor 1 subunit alpha (HIF1a) signaling, and neuropathic pain signaling (FIG. 6). Pulmonary fibrosis occurs when the lung is being damaged in a manner that promotes tissue scaring and the loss of lung function^{36,37}. In the case of Idiopathic pulmonary fibrosis (IPF), the cause of disease remains unknown and treatment strategies are desperately needed with patient survival averaging between 3-5 years post-diagnosis^{37,38}. Interestingly, HIF1a activity is driven by reductions in oxygen levels and is triggered in AMs as a result of lung fibrosis³⁹. Finally, neuropathic pain signaling is associated with disease states that develop spontaneously and are not associated with the actions of external stimuli⁴⁰. Collectively, these data demonstrate that AMs require Car4 expression to prevent the induction of profibrotic pathways and idiopathic inflammation.

### Car4 Operates as an Important Regulator of Antihelminth Macrophage Responses

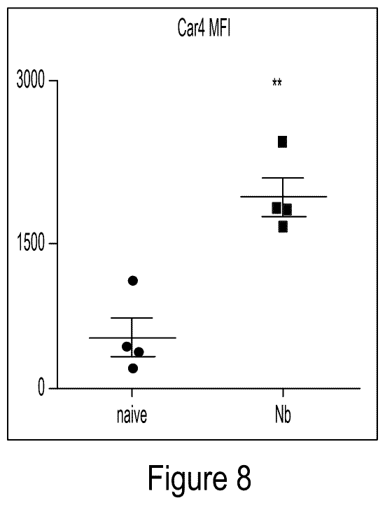

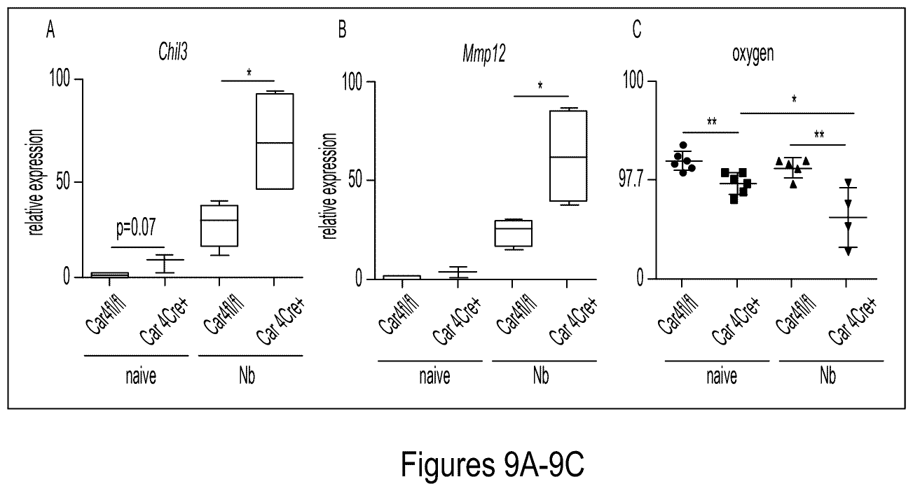

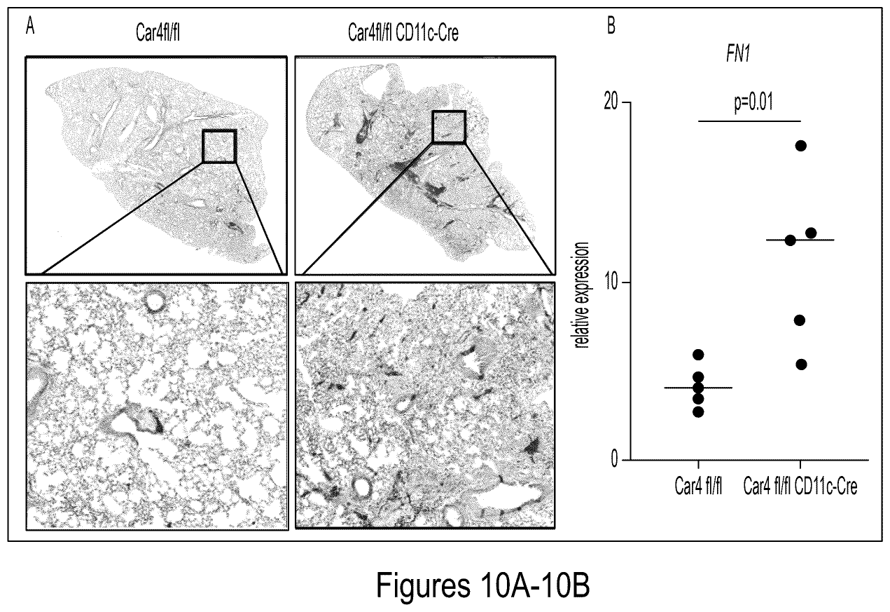

The studies presented above demonstrate that deletion of Car4 in AMs results in the induction of idiopathic lung inflammation. Moreover, the data reveal that Car4-deficient AMs have distinct transcriptional profiles that are consistent with increased inflammatory responses and fibrosis (FIG. 6,7). Additional analysis of these transcriptional profiles also demonstrated that Car4-deficient AMs exhibit characteristics of M2 macrophages, including increased expression of the genes encoding Arginase 1 (Arg1), Relmα (Retnla) and Macrophage galactose-type C-type lectin 2 (Mgl2) (FIG. 7) 12,41. M2 macrophages are known to promote matrix deposition and tissue remodeling, and their presence is associated with lung fibrosis^{11,13}. Collectively, these data provoke the hypothesis that Car4 operates to negatively regulate M2 responses and thereby prevents excessive tissue fibrosis. To better investigate this possibility, mice were infected with the hookworm Nippostringlylus brasilinesis (Nb) and Car4 expression by AMs was monitored. As part of its lifecycle, Nb passages through the lungs of its host; and it is well established that AMs with M2 phenotypes play critical roles in promoting worm clearance and initiating wound healing responses needed to maintain lung function^{23,42}. Consistent with a role for Car4 in regulating M2 responses, a significantly increased expression of Car4 by AMs was observed following an Nb challenge (FIG. 8). Next, control and Car4-AM^{-/-} mice were infected with Nb and M2 responses (markers of tissue remodeling and lung function) were monitored. Consistent with the present overarching hypothesis, Car4-AM mice infected with Nb exhibited significantly increased expression of the M2-associated marker Chil3 and the fibrosis-associated molecule matrix metalloproteinase 12 (Mmp12) 43,44. Additionally, infected Car4-AM^{-/-} mice presented with dramatically reduced oxygen levels (FIG. 9A-C). These exciting results are indicative of excessively potent tissue remodeling that causes an unwanted loss of tissue function. Further, these data highlight the importance of AMs in properly balancing helminth-induced inflammation to protect the integrity of the lung. Collectively, these findings identify a previously unappreciated regulator of AM activation that operates to reduce fibrosis following an infectious challenge. To better evaluate this, the lungs of Nb infected WT and Car4-AM^{-/-} mice were evaluated for features of fibrosis. Car4-AM^{-/-} mice infected with Nb exhibited dramatically increased trichrome staining and expression of fibronectin-1 (FN1) (FIG. 10), well established markers of lung fibrosis⁴⁵. Collectively, these data further support the hypothesis that Car4 operates to negatively regulate profibrotic responses in the lung.

### Car4 Down Regulated in Human Idiopathic Pulmonary Fibrosis (IPF)

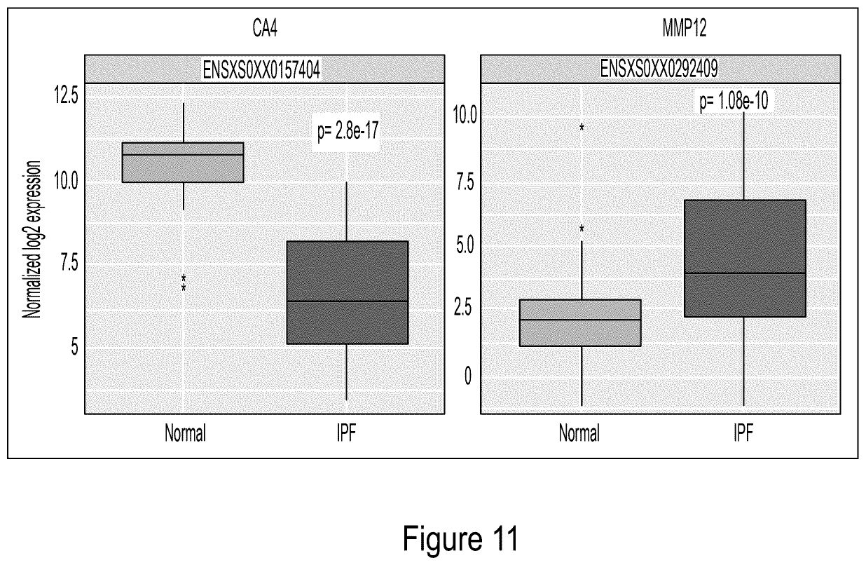

The murine studies presented herein have also shown that the deletion of Car4 on AMs results in spontaneous inflammation, increases expression of MMP12, and reduced lung function. IPF is a lung disease that is characterized by spontaneous lung inflammation, tissue fibrosis, and reduced lung function. IPF is a chronic condition that worsens over time, and, unfortunately, patients suffering from IPF have an average survival rate of 3-5 years^{37,38}. Therapeutic approaches to treat IPF are extremely limited, largely because of the lack of knowledge regarding the underlying cause(s) of IPF. Therefore, it is critical to gain more mechanistic insight into the causes of IPF to inform much needed therapeutic strategies. Given that the animal model presented herein appears to mimic features of human IPF, an investigation was undertaken to determine if Car4 is downregulated in the lungs of IPF patients compared to otherwise health controls. To test this, publicly available data that was uploaded into the GEO omnibus (accession number GSE134692) was accessed. In this study, large scale RNA-seq analysis of lung tissue from transplant stage IPF patients (46 samples) was performed and compared those to transcriptional profiles to non-diseased lung tissue (26 samples). Remarkably, human carbonic anhydrase 4 (CA4) was one of the most significantly down regulated genes in IPF patients compared to controls (p=2.8e-17). Consistent the animal model presented herein, IPF patients also presented with significantly upregulated MMP12 (p=1.08e-10) compared to controls (FIG. 11). These data demonstrate that reduced CA4 expression is associated with IPF, illustrate that the presently disclosed animal model recapitulates aspects of human disease, and highlight the important clinical potential of targeting Carbonic anhydrase 4 in treating lung fibrosis.

### Car4 can be Delivered Therapeutically to Alleviate Lung Pathology in a Murine Model of Lung Fibrosis

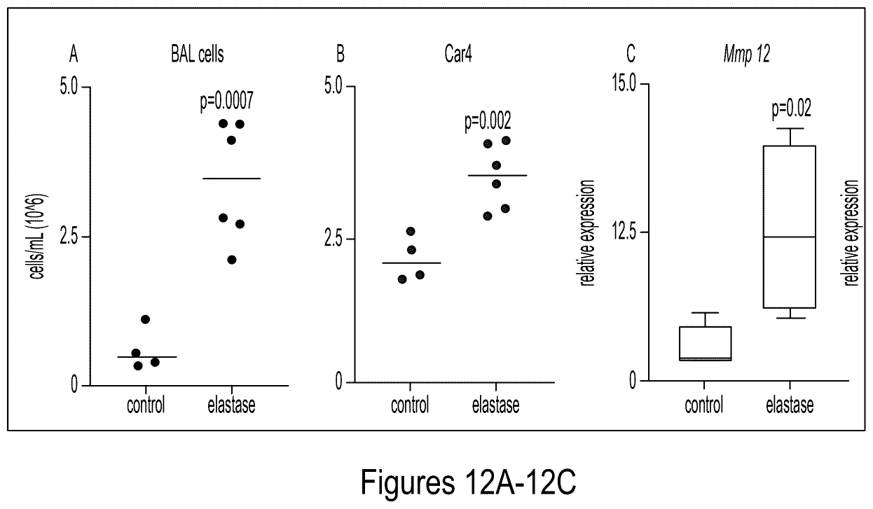

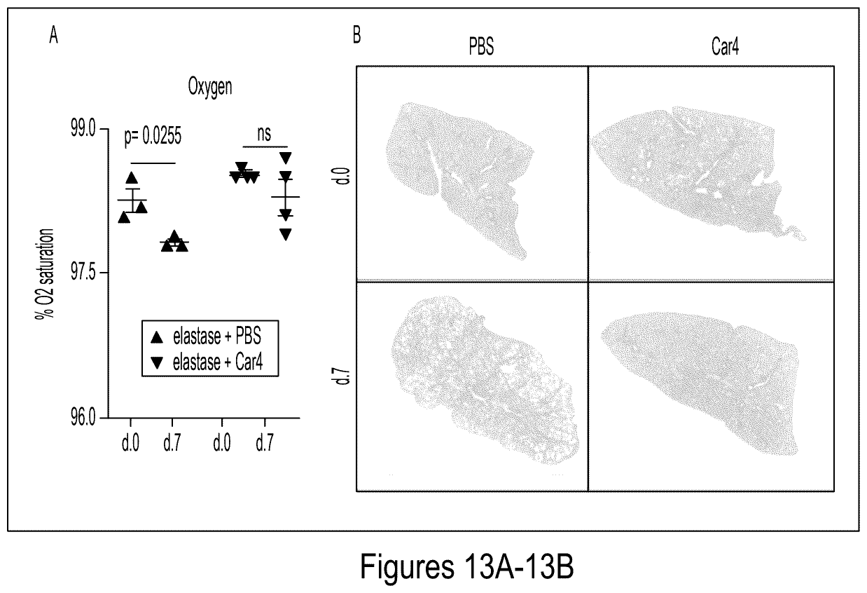

In addition to infections, lung inflammation and tissue remodeling can be caused by the inhalation of noninfectious agents such as toxins, irritants, or particulate matter^{2,36}. Thus, it is important to evaluate whether Car4 also regulates AM activation and pulmonary inflammation in response to noninfectious stimuli. Instillation of elastase into the airways is a well-established model of pulmonary inflammation that induces emphysematous-like pathology and pulmonary fibrosis⁴⁶. Therefore, an investigation was undertaken to determine whether the elastase model mimics aspects of the inflammatory response observed at baseline and post-Nb in Car4-AM^{-/-} mice. Importantly, instillation of elastase into mice, as described previously, resulted in lung inflammation as determined by the cellular content of the BAL and significantly increased expression of Car4 by AMs (FIG. 12A,B)⁴⁶. Further, elastase treatment also resulted in significantly increased expression of Mmp12 (FIG. 12C). Next, an investigation was undertaken to test whether treatment with Car4 was sufficient to alter elastase induced lung pathology. Remarkably, intratracheal administration of recombinant Car4 was sufficient to prevent elastase-induced reduction in peripheral O2 levels, as well as to alleviate elastase-induced lung pathology (FIG. 13). Collectively, these data suggest that Car4 represents a novel therapeutic target for the treatment of lung fibrosis.

### Materials and Methods

### Mice

BL/6 mice and CD11c-Cre were purchased from The Jackson Laboratory (Bar Harbor, ME) and Car4-floxed mice were generated at the Rutgers New Jersey Medical School Genome Editing Shared Resource. All mice were bred and maintained in a specific pathogen-free, virus Ab-free facility at Rutgers New Jersey Medical School Comparative Medicine Resources. Healthy 8-12 week old mice were selected for treatment groups from purchased or bred colonies. The studies have been reviewed and approved by the Institutional Animal Care and Use Committee at Rutgers—the State University of New Jersey. The experiments herein were conducted according to the principles set forth in the Guide for the Care and Use of Laboratory Animals, Institute of Animal Resources, National Research Council, Department of Health, Education and Welfare (US National Institutes of Health).

### Parasite Culture and Inoculation of Mice

N. brasiliensis (NB) L3 were maintained in a petri dish culture containing charcoal and sphagnum moss. The larvae were isolated from cultures using a modified Baermann apparatus with 400U penicillin, 400 μg ml⁻¹ streptomycin, and 400 μg ml⁻¹ Neomycin (GIBCO, Rockville, MD) in sterile PBS, and then washed with sterile PBS three times. Mice were infected subcutaneously with a 40 μl suspension of 650 Nb L3.

### Flow Cytometry and Adoptive Transfer

Lung tissue was washed with stirring at room temperature for 10 min in Hank's balanced salt solution (HBSS) with 1.3 mM EDTA (Invitrogen), then minced and treated at 37° C. for 30 minutes with collagenase (1 mg/ml; Sigma) in RPMI1640 with 10% fetal calf serum (FCS) and with 100 μg/ml of DNase for 10 minutes. Cells were lysed with ACK (Lonza, Walkersville, MD) to remove erythrocytes. Cells were blocked with Fc Block (BD Biosciences, San Jose, CA), directly stained with fluorochrome-conjugated antibodies against CD45, F4/80, CD64, CD11c, Siglec-F, CD11b, Ly6C, and analyzed by flow cytometry for alveolar macrophages (AMS) (F4/80⁺CD64⁺SiglecF⁺CD11c^{hi}).

### Cytokine Gene Expression by RT-PCR

For qPCR, RNA was extracted from lung tissue or sorted MA and reverse transcribed to cDNA. qPCR was done with Taqman (Life Technologies Corporation, Carlsbad, CA) kits and the Applied Biosystems 7500 Real-Time PCR System. All data were normalized to 18S ribosomal RNA, and the quantification of differences between treatment groups was calculated according to the manufacturer's instructions. Gene expression is presented as the fold increase over naïve WT controls.

### Pulse Oximetry

Oxygen saturation was evaluated with the MouseOx Plus® (Starr Lifesciences Corp) following manufacturer's instructions. Briefly, the hair around the thigh was removed 1 day before Nb infection. Then, mice were anesthetized with 5% isoflurane and oxygen saturation was monitored using the thigh sensor for an interval of approximately 5 minutes. This time point was used to collect representative and error-free data.

### RNA Sequencing

Alveolar macrophages were sort-purified by gating on CD45+, CD64+, F4/80+, CD11c+, SiglecF+ and submitted for RNA-seq analysis at the Rutgers New Jersey Medical School Genomics Center. Cells from a minimum of three biological replicates were sorted and pooled. The purity of all cell populations was 98% or greater. RNA was extracted using the RNeasy Plus Micro Kit (catalog no. 74034; QIAGEN). Illumina-compatible libraries were generated using the NEBNext Ultra II DNA Library Prep Kit for Illumina (catalog no. E7645S; New England BioLabs) and sequenced using an Illumina NovaSeq 6000 system in a paired-end 2×50-base pair (bp) reads configuration. Bulk RNA-seq analysis was performed in accordance with the nf-core RNA-seq guidelines v.1.4.2⁴⁷. Briefly, the output reads were aligned to the GRCm38 (mm10) genome using STAR, followed by gene count generation using feature Counts and StringTie⁴⁸⁻⁵⁰. Read counts were normalized and compared between groups for differential gene expression using DESeq2 with significance cutoff at false discovery rate-adjusted p<0.0551. Determination of functional pathways was performed using Ingenuity Pathway Analysis (IPA) on differentially expressed genes.

### Elastase-Induced Fibrosis Mouse Model

BL/6 mice at the age of 8-12 weeks were intratracheally treated with 1.5U porcine pancreatic elastase (Sigma, 324682-250U) in 50 μl of PBS or PBS alone while anesthetized under isoflurane. In selected experiments, mice were additionally treated with 0.72pmol/min activity (0.24 ug) recombinant mouse carbonic anhydrase IV protein in 20 μl PBS (R&D Systems, 2414-CA-010) every other day starting day-1 of elastase treatment.

### Cited Documents

1. Collaborators GBDCRD. Prevalence and attributable health burden of chronic respiratory diseases, 1990-2017: a systematic analysis for the Global Burden of Disease Study 2017. Lancet Respir Med. 2020; 8 (6): 585-96. Epub 2020 Jun. 12. doi: 10.1016/S2213-2600 (20) 30105-3. PubMed PMID: 32526187; PMCID: PMC7284317.
2. Societies FoIR. The Global Impact of Respiratory Disease-Second Addition. Sheffield, European Respiratory Society. 2017; ISBN: 9781849840837.
3. George P M, Wells A U, Jenkins R G. Pulmonary fibrosis and COVID-19: the potential role for antifibrotic therapy. Lancet Respir Med. 2020; 8 (8): 807-15. Epub 2020 May 19. doi: 10.1016/S2213-2600 (20) 30225-3. PubMed PMID: 32422178; PMCID: PMC7228727.
4. Halpin DMG, Criner G J, Papi A, Singh D, Anzueto A, Martinez F J, Agusti A A, Vogelmeier C F. Global Initiative for the Diagnosis, Management, and Prevention of Chronic Obstructive Lung Disease. The 2020 GOLD Science Committee Report on COVID-19 and Chronic Obstructive Pulmonary Disease. Am J Respir Crit Care Med. 2021; 203 (1): 24-36. Epub 2020 Nov. 5. doi: 10.1164/rccm.202009-3533S O. PubMed PMID: 33146552; PMCID: PMC7781116.
5. Kulikauskaite J, Wack A. Teaching Old Dogs New Tricks? The Plasticity of Lung Alveolar Macrophage Subsets. Trends Immunol. 2020; 41 (10): 864-77. Epub 2020 Sep. 9. doi: 10.1016/j.it.2020.08.008. PubMed PMID: 32896485; PMCID: PMC7472979.
6 Hussell T, Bell T J. Alveolar macrophages: plasticity in a tissue-specific context. Nat Rev Immunol. 2014; 14 (2): 81-93. Epub 2014 Jan. 22. doi: 10.1038/nri3600. PubMed PMID: 24445666.
7. Hu G, Christman J W. Editorial: Alveolar Macrophages in Lung Inflammation and Resolution. Front Immunol. 2019; 10:2275. Epub 2019 Oct. 17. doi: 10.3389/fimmu.2019.02275. PubMed PMID: 31616438; PMCID: PMC6768960.
8. Lavin Y, Winter D, Blecher-Gonen R, David E, Keren-Shaul H, Merad M, Jung S, Amit I. Tissue-resident macrophage enhancer landscapes are shaped by the local microenvironment. Cell. 2014; 159 (6): 1312-26. Epub 2014 Dec. 7. doi: 10.1016/j.cell.2014.11.018. PubMed PMID: 25480296; PMCID: PMC4437213.
9. Yu X, Buttgereit A, Lelios I, Utz S G, Cansever D, Becher B, Greter M. The Cytokine TGF-beta Promotes the Development and Homeostasis of Alveolar Macrophages. Immunity. 2017; 47 (5): 903-12 e4. Epub 2017 Nov. 12. doi: 10.1016/j.immuni.2017.10.007. PubMed PMID: 29126797.
10. Kreider T, Anthony R M, Urban J F, Jr., Gause W C. Alternatively activated macrophages in helminth infections. Curr Opin Immunol. 2007; 19 (4): 448-53. Epub 2007 Aug. 19. doi: 10.1016/j.coi.2007.07.002. PubMed PMID: 17702561; PMCID: PMC2000338.
11. Allen J E, Sutherland T E. Host protective roles of type 2 immunity: parasite killing and tissue repair, flip sides of the same coin. Semin Immunol. 2014; 26 (4): 329-40. Epub 2014 Jul. 17. doi: 10.1016/j.smim.2014.06.003. PubMed PMID: 25028340; PMCID: PMC4179909.
12. Mosser D M, Edwards J P. Exploring the full spectrum of macrophage activation. Nat Rev Immunol. 2008; 8 (12): 958-69. Epub 2008 Nov. 26. doi: 10.1038/nri2448. PubMed PMID: 19029990; PMCID: PMC2724991.
13. Wynn T A, Vannella K M. Macrophages in Tissue Repair, Regeneration, and Fibrosis. Immunity. 2016; 44 (3): 450-62. Epub 2016 Mar. 18. doi: 10.1016/j.immuni.2016.02.015. PubMed PMID: 26982353; PMCID: PMC4794754.
14. Cox F E. History of human parasitology. Clin Microbiol Rev. 2002; 15 (4): 595-612. Epub 2002 Oct. 5. PubMed PMID: 12364371; PMCID: 126866.
15. Gause W C, Wynn T A, Allen J E. Type 2 immunity and wound healing: evolutionary refinement of adaptive immunity by helminths. Nature reviews Immunology. 2013; 13 (8): 607-14. Epub 2013 Jul. 6. doi: 10.1038/nri3476. PubMed PMID: 23827958; PMCID: 3789590.
16. Pulendran B, Artis D. New paradigms in type 2 immunity. Science. 2012; 337 (6093): 431-5. Epub 2012 Jul. 28. doi: 10.1126/science.1221064. PubMed PMID: 22837519.
17. Craig J M, Scott A L. Helminths in the lungs. Parasite Immunol. 2014; 36 (9): 463-74. Epub 2014 Sep. 10. doi: 10.1111/pim.12102. PubMed PMID: 25201409.
18. Maizels R M, Hewitson J P, Smith K A. Susceptibility and immunity to helminth parasites. Current opinion in immunology. 2012; 24 (4): 459-66. Epub 2012 Jul. 17. doi: 10.1016/j.coi.2012.06.003. PubMed PMID: 22795966; PMCID: 3437973.
19. Allen J E, Sutherland T E. Host protective roles of type 2 immunity: parasite killing and tissue repair, flip sides of the same coin. Semin Immunol. 2014; 26 (4): 329-40. Epub 2014 Jul. 17. doi: 10.1016/j.smim.2014.06.003. PubMed PMID: 25028340; PMCID: 4179909.
20. Davies L C, Jenkins S J, Allen J E, Taylor P R. Tissue-resident macrophages. Nat Immunol. 2013; 14 (10): 986-95. Epub 2013 Sep. 21. doi: 10.1038/ni.2705. PubMed PMID: 24048120; PMCID: 4045180.
21. Gundra U M, Girgis N M, Gonzalez M A, San Tang M, Van Der Zande HJP, Lin J D, Ouimet M, Ma L J, Poles J, Vozhilla N, Fisher E A, Moore K J, Loke P. Vitamin A mediates conversion of monocyte-derived macrophages into tissue-resident macrophages during alternative activation. Nat Immunol. 2017; 18 (6): 642-53. Epub 2017 Apr. 25. doi: 10.1038/ni.3734. PubMed PMID: 28436955; PMCID: 5475284.
22. Okabe Y, Medzhitov R. Tissue-specific signals control reversible program of localization and functional polarization of macrophages. Cell. 2014; 157 (4): 832-44. Epub 2014 May 6. doi: 10.1016/j.cell.2014.04.016. PubMed PMID: 24792964; PMCID: 4137874.
23. Chen F, El-Naccache D W, Ponessa J J, Lemenze A, Espinosa V, Wu W, Lothstein K, Jin L, Antao O, Weinstein J S, Damani-Yokota P, Khanna K, Murray P J, Rivera A, Siracusa M C, Gause W C. Helminth resistance is mediated by differential activation of recruited monocyte-derived alveolar macrophages and arginine depletion. Cell Rep. 2022; 38 (2): 110215. Epub 2022 Jan. 13. doi: 10.1016/j.celrep.2021.110215. PubMed PMID: 35021079.
24. Boone C D, Habibzadegan A, Gill S, McKenna R. Carbonic anhydrases and their biotechnological applications. Biomolecules. 2013; 3 (3): 553-62. Epub 2013 Jan. 1. doi: 10.3390/biom3030553. PubMed PMID: 24970180; PMCID: 4030944.
25. Henry E K, Sy C B, Inclan-Rico J M, Espinosa V, Ghanny S S, Dwyer D F, Soteropoulos P, Rivera A, Siracusa M C. Carbonic anhydrase enzymes regulate mast cell-mediated inflammation. J Exp Med. 2016; 213 (9): 1663-73. Epub 2016 Aug. 17. doi: 10.1084/jem.20151739. PubMed PMID: 27526715; PMCID: PMC4995079.
26. Inclan-Rico J M, Hernandez C M, Henry E K, Federman H G, Sy C B, Ponessa J J, Lemenze A D, Joseph N, Soteropoulos P, Beaulieu A M, Yap G S, Siracusa M C. Trichinella spiralis-induced mastocytosis and erythropoiesis are simultaneously supported by a bipotent mast cell/erythrocyte precursor cell. PLOS Pathog. 2020; 16 (5): e1008579. Epub 2020 May 19. doi: 10.1371/journal.ppat. 1008579. PubMed PMID: 32421753; PMCID: PMC7259795.
27. Xu J, Xu X, Wang B, Ma Y, Zhang L, Xu H, Hu Y, Wu J, Cao X. Nuclear carbonic anhydrase 6B associates with PRMT5 to epigenetically promote IL-12 expression in innate response. Proc Natl Acad Sci USA. 2017; 114 (32): 8620-5. Epub 2017 Jul. 26. doi: 10.1073/pnas. 1700917114. PubMed PMID: 28739930; PMCID: PMC5559001.
28. Henry E K, Sy C B, Inclan-Rico J M, Espinosa V, Ghanny S S, Dwyer D F, Soteropoulos P, Rivera A, Siracusa M C. Carbonic anhydrase enzymes regulate mast cell-mediated inflammation. J Exp Med. 2016; 213 (9): 1663-73. Epub 2016 Aug. 17. doi: 10.1084/jem.20151739. PubMed PMID: 27526715; PMCID: 4995079.
29. Todd E M, Zhou J Y, Szasz T P, Deady L E, D'Angelo J A, Cheung M D, Kim A H, Morley S C. Alveolar macrophage development in mice requires L-plastin for cellular localization in alveoli. Blood. 2016; 128 (24): 2785-96. Epub 2016 Oct. 21. doi: 10.1182/blood-2016-03-705962. PubMed PMID: 27758872; PMCID: PMC5159703.
30. Yao Y, Liu Q, Adrianto I, Wu X, Glassbrook J, Khalasawi N, Yin C, Yi Q, Dong Z, Geissmann F, Zhou L, Mi Q S. Histone deacetylase 3 controls lung alveolar macrophage development and homeostasis. Nat Commun. 2020; 11 (1): 3822. Epub 2020 Aug. 1. doi: 10.1038/s41467-020-17630-6. PubMed PMID: 32732898; PMCID: PMC7393351.
31. Lee C G, Homer R J, Zhu Z, Lanone S, Wang X, Koteliansky V, Shipley J M, Gotwals P, Noble P, Chen Q, Senior R M, Elias J A. Interleukin-13 induces tissue fibrosis by selectively stimulating and activating transforming growth factor beta (1). J Exp Med. 2001; 194 (6): 809-21. Epub 2001 Sep. 19. doi: 10.1084/jem. 194.6.809. PubMed PMID: 11560996; PMCID: PMC2195954.
32. Borthwick L A, Barron L, Hart K M, Vannella K M, Thompson R W, Oland S, Cheever A, Sciurba J, Ramalingam T R, Fisher A J, Wynn T A. Macrophages are critical to the maintenance of IL-13-dependent lung inflammation and fibrosis. Mucosal Immunol. 2016; 9 (1): 38-55. Epub 2015 Apr. 30. doi: 10.1038/mi.2015.34. PubMed PMID: 25921340; PMCID: PMC4626445.
33. Passalacqua G, Mincarini M, Colombo D, Troisi G, Ferrari M, Bagnasco D, Balbi F, Riccio A, Canonica G W. IL-13 and idiopathic pulmonary fibrosis: Possible links and new therapeutic strategies. Pulm Pharmacol Ther. 2017; 45:95-100. Epub 2017 May 16. doi: 10.1016/j.pupt.2017.05.007. PubMed PMID: 28501346.
34. Park M J, Moon S J, Lee E J, Jung K A, Kim E K, Kim D S, Lee J H, Kwok S K, Min J K, Park S H, Cho M L. IL-1-IL-17 Signaling Axis Contributes to Fibrosis and Inflammation in Two Different Murine Models of Systemic Sclerosis. Front Immunol. 2018; 9:1611. Epub 2018 Jul. 26. doi: 10.3389/fimmu.2018.01611. PubMed PMID: 30042768; PMCID: PMC6048384.
35. Fabre T, Molina M F, Soucy G, Goulet J P, Willems B, Villeneuve J P, Bilodeau M, Shoukry N H. Type 3 cytokines IL-17A and IL-22 drive TGF-beta-dependent liver fibrosis. Sci Immunol. 2018; 3 (28). Epub 2018 Oct. 28. doi: 10.1126/sciimmunol.aar7754. PubMed PMID: 30366940.
36. Wijsenbeek M, Cottin V. Spectrum of Fibrotic Lung Diseases. N Engl J Med. 2020; 383 (10): 958-68. Epub 2020 Sep. 3. doi: 10.1056/NEJMra2005230. PubMed PMID: 32877584.
37. Martinez F J, Collard H R, Pardo A, Raghu G, Richeldi L, Selman M, Swigris J J, Taniguchi H, Wells A U. Idiopathic pulmonary fibrosis. Nat Rev Dis Primers. 2017; 3:17074. Epub 2017 Oct. 21. doi: 10.1038/nrdp.2017.74. PubMed PMID: 29052582.
38. Richeldi L, Collard H R, Jones M G. Idiopathic pulmonary fibrosis. Lancet. 2017; 389 (10082): 1941-52. Epub 2017 Apr. 4. doi: 10.1016/S0140-6736 (17) 30866-8. PubMed PMID: 28365056.
39. Majmundar A J, Wong W J, Simon M C. Hypoxia-inducible factors and the response to hypoxic stress. Mol Cell. 2010; 40 (2): 294-309. Epub 2010 Oct. 23. doi: 10.1016/j.molcel.2010.09.022. PubMed PMID: 20965423; PMCID: PMC3143508.
40. Baron R. Mechanisms of disease: neuropathic pain—a clinical perspective. Nat Clin Pract Neurol. 2006; 2 (2): 95-106. Epub 2006 Aug. 26. doi: 10.1038/ncpneuro0113. PubMed PMID: 16932531.
41. Siracusa M C, Reece J J, Urban J F, Jr., Scott A L. Dynamics of lung macrophage activation in response to helminth infection. J Leukoc Biol. 2008; 84 (6): 1422-33. Epub 2008 Aug. 23. doi: 10.1189/jlb.0308199. PubMed PMID: 18719016; PMCID: PMC2614596.
42. Chen F, Liu Z, Wu W, Rozo C, Bowdridge S, Millman A, Van Rooijen N, Urban J F, Jr., Wynn T A, Gause W C. An essential role for TH2-type responses in limiting acute tissue damage during experimental helminth infection. Nature medicine. 2012; 18 (2): 260-6. Epub 2012 Jan. 17. doi: 10.1038/nm.2628. PubMed PMID: 22245779; PMCID: 3274634.
43. Matute-Bello G, Wurfel M M, Lee J S, Park D R, Frevert C W, Madtes D K, Shapiro S D, Martin T R. Essential role of MMP-12 in Fas-induced lung fibrosis. Am J Respir Cell Mol Biol. 2007; 37 (2): 210-21. Epub 2007 Apr. 21. doi: 10.1165/rcmb.2006-0471O C. PubMed PMID: 17446527; PMCID: PMC1976544.
44. Pardo A, Cabrera S, Maldonado M, Selman M. Role of matrix metalloproteinases in the pathogenesis of idiopathic pulmonary fibrosis. Respir Res. 2016; 17:23. Epub 2016 Mar. 6. doi: 10.1186/s12931-016-0343-6. PubMed PMID: 26944412; PMCID: PMC4779202.
45. Tsukui T, Sun K H, Wetter J B, Wilson-Kanamori J R, Hazelwood L A, Henderson N C, Adams T S, Schupp J C, Poli S D, Rosas I O, Kaminski N, Matthay M A, Wolters P J, Sheppard D. Collagen-producing lung cell atlas identifies multiple subsets with distinct localization and relevance to fibrosis. Nat Commun. 2020; 11 (1): 1920. Epub 2020 Apr. 23. doi: 10.1038/s41467-020-15647-5. PubMed PMID: 32317643; PMCID: PMC7174390 Abbvie, Pfizer and Pliant Therapeutics. He serves on the Scientific Review Board for Genentech, xCella Biosciences and Optikira. M.A.M. has received research funding from Bayer Therapeutics and is a consultant for Cerus Therapeutics and Genle Life Sciences. T.S.A., J.C.S., S.P., I.O.R., and N.K. are inventors on a provisional patent application (62/849,644), Yale University and Brigham and Women's Hospital, Inc. that covers methods related to IPF associated cell subsets. N.K. served as a consultant to Biogen Idec, Boehringer Ingelheim, Third Rock, Pliant, Samumed, NuMedii, Indaloo, Theravance, LifeMax, Three Lake Partners, Optikira over the last 3 years and received non-financial support from MiRagen.
46. Shibata S, Miyake K, Tateishi T, Yoshikawa S, Yamanishi Y, Miyazaki Y, Inase N, Karasuyama H. Basophils trigger emphysema development in a murine model of COPD through IL-4-mediated generation of MMP-12-producing macrophages. Proc Natl Acad Sci USA. 2018; 115 (51): 13057-62. Epub 2018 Dec. 5. doi: 10.1073/pnas.1813927115. PubMed PMID: 30510003; PMCID: PMC6305004.
47. Ewels P A, Peltzer A, Fillinger S, Patel H, Alneberg J, Wilm A, Garcia M U, Di Tommaso P, Nahnsen S. The nf-core framework for community-curated bioinformatics pipelines. Nat Biotechnol. 2020; 38 (3): 276-8. Epub 2020 Feb. 15. doi: 10.1038/s41587-020-0439-x. PubMed PMID: 32055031.
48. Liao Y, Smyth G K, Shi W. featureCounts: an efficient general purpose program for assigning sequence reads to genomic features. Bioinformatics. 2014; 30 (7): 923-30. Epub 2013 Nov. 15. doi: 10.1093/bioinformatics/btt656. PubMed PMID: 24227677.
49. Pertea M, Pertea G M, Antonescu C M, Chang T C, Mendell J T, Salzberg S L. StringTie enables improved reconstruction of a transcriptome from RNA-seq reads. Nat Biotechnol. 2015; 33 (3): 290-5. Epub 2015 Feb. 19. doi: 10.1038/nbt.3122. PubMed PMID: 25690850; PMCID: PMC4643835.
50. Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013; 29 (1): 15-21. Epub 2012 Oct. 30. doi: 10.1093/bioinformatics/bts635. PubMed PMID: 23104886; PMCID: PMC3530905.
51. Love M I, Huber W, Anders S. Moderated estimation of fold change and dispersion for RNA-seq data with DESeq2. Genome Biol. 2014; 15 (12): 550. Epub 2014 Dec. 18. doi: 10.1186/s13059-014-0550-8. PubMed PMID: 25516281; PMCID: PMC4302049.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating lung fibrosis and/or emphysema comprising administering to a mammal in need thereof an effective amount of at least one of a carbonic anhydrase 4 (Car4) enzyme, a nucleic acid encoding a Car4 enzyme, or a Car4 enzyme activator.

2. The method of claim 1, wherein the Car4 enzyme is administered.

3. The method of claim 2, wherein the Car4 enzyme comprises an amino acid sequence having at least 80% sequence identity to an amino acid sequence of any one of SEQ ID NOs: 1 to 3.

4. The method of claim 2, wherein the Car4 enzyme comprises an amino acid sequence having at least 90% sequence identity to an amino acid sequence of any one of SEQ ID NOs: 1 to 3.

5. The method of claim 2, wherein the Car4 enzyme comprises an amino acid sequence having at least 95% sequence identity to an amino acid sequence of any one of SEQ ID NOs: 1 to 3.

6. The method of claim 1, wherein the nucleic acid encoding a Car4 enzyme is administered.

7. The method of claim 6, wherein the nucleic acid encoding a Car4 enzyme is comprised within an expression cassette comprising a promoter, and wherein the nucleic acid is operably linked to the promoter.

8. The method of claim 7, wherein the expression cassette is comprised within a vector (e.g., a viral vector).

9. The method of claim 6, wherein the nucleic acid encoding a Car4 enzyme comprises a nucleic acid sequence having at least 80% sequence identity to SEQ ID NOs: 4 or 5.

10. The method of claim 6, wherein the nucleic acid encoding a Car4 enzyme comprises a nucleic acid sequence having at least 85% sequence identity to SEQ ID NOs: 4 or 5.

11. The method of claim 1, wherein the Car4 enzyme activator is administered.

12. The method of claim 10, wherein the Car4 enzyme activator comprises at least one of L-phenylalanine, D-phenylalanine, L-histidine, D-histidine, or a pharmaceutically acceptable salt thereof.

13. The method of claim 1, further comprising administering one or more additional therapeutic agents or therapies to the mammal.

14. The method of claim 13, wherein the one or more additional therapeutic agents or therapies is an anti-inflammatory agent, a corticosteroid, an anticholinergic, a beta2-adrenergic agonist, and/or an agent useful for treating fibrosis (e.g., Nintedanib or Pirfenidone).

15. The method of claim 1, wherein the lung fibrosis and/or emphysema is M2 macrophage-mediated.

16. The method of claim 1, which is a method for treating lung fibrosis wherein the lung fibrosis is idiopathic pulmonary fibrosis (IPF).

17. The method of claim 1, which is a method of treating emphysema, wherein the mammal has chronic obstructive pulmonary disease (COPD).

18. The method of claim 1, wherein the lung fibrosis and/or emphysema is caused by COVID-19.

19. The method of claim 1, wherein the lung fibrosis and/or emphysema is caused by a parasite infection.

20. The method of claim 1, wherein the lung fibrosis and/or emphysema is caused by inhalation of noninfectious agents including at least one of a toxin, an irritant, or particulate matter.

21. The method of claim 1, wherein the at least one of the Car4 enzyme, the nucleic acid encoding a Car4 enzyme, the Car4 enzyme activator is administered intratracheally, by inhalation, by nebulization, nasally, or intravenously.

22. The method of claim 1, wherein the at least one of the Car4 enzyme, the nucleic acid encoding a Car4 enzyme, the Car4 enzyme activator is comprised in a pharmaceutical composition that comprises a pharmaceutically acceptable carrier.

23. A kit comprising at least one of a Car4 enzyme, a nucleic acid encoding a Car4 enzyme, or a Car4 enzyme activator; packaging material; and instructions for administering the at least one of a Car4 enzyme, a nucleic acid encoding a Car4 enzyme, or a Car4 enzyme activator to a mammal in need thereof to treat lung fibrosis and/or emphysema.

24. (canceled)

25. (canceled)

26. The method, enzyme or use of claim 1, wherein the mammal is human.
